(19) United States
(12) Patent Application Publication
(10) Pub. No.: US 2020/0197268 A1
(43) Pub. Date: Jun. 25, 2020

(54) Stable Stannous Oral Care Product

(71) Applicant: Colgate-Palmolive Company, New York, NY (US)

(72) Inventors: Carl MYERS, Wayne, NJ (US); Iraklis PAPPAS, Pennsauken, NJ (US); Zhigang HAO, Bridgewater, NJ (US); Saide TANG, Princeton, NJ (US)

(73) Assignee: Colgate-Palmolive Company, New York, NY (US)

(21) Appl. No.: 16/717,014

(22) Filed: Dec. 17, 2019

Related U.S. Application Data
(60) Provisional application No. 62/784,253, filed on Dec. 21, 2018.
(60) Provisional application No. 62/861,679, filed on Jun. 14, 2019.

(51) Int. Cl. A61K 8/21; A61K 8/365; A61K 8/27; A61K 8/34; A61K 8/24; A61Q 11/00

(52) U.S. Cl. CPC A61K 8/21; A61K 8/365; A61K 8/27; A61K 2800/52; A61K 8/24; A61Q 11/00; A61K 8/345

(57) ABSTRACT

Disclosed herein are high water oral care compositions comprising a stannous ion source, which compositions have improved stability. Methods of making and using the compositions are also provided.

### FIELD OF THE DISCLOSURE

This disclosure relates to high water oral care compositions comprising a stannous salt in a stabilizing base providing a reservoir of orthophosphate ion. Methods of making and using the compositions are also provided.

### BACKGROUND

Stannous salts, such as stannous fluoride (SnF₂) and stannous chloride (SnCl₂), have been used in oral care products for many years, as the stannous ion has antibacterial properties, and so is useful to treat conditions like gingivitis. Oral care formulations comprising stannous salts, however, have been limited by the instability of stannous salts in aqueous solutions. Stannous salts readily hydrolyze above pH 4, resulting in precipitation from solution, with a consequent loss of the therapeutic properties, and the stannous ion (Sn²⁺) readily forms stannous oxy-fluoride or oxidizes to form stannic (Sn⁴⁺) compounds, which may stain the teeth, reduce or inhibit enamel fluoridation, and eliminate the antibacterial effects of the stannous fluoride. Moreover, the stannic compounds may impart an unpleasant taste and gritty feel to the formulations. To avoid the degradation of stannous salts in aqueous oral care formulations, and in particular to avoid oxidation of the stannous ion to form undesirable stannic compounds, commercial oral care formulations containing stannous salts generally have low amounts of water (e.g., less than 10%) and/or have chelating agents to sequester and protect the stannous ion. Low water formulations, however, may have higher manufacturing costs and/or undesirable organoleptic properties, and chelating agents may chelate and reduce the availability and effectiveness of beneficial cations such as zinc ions, as well as the stannous ions.

There is a need for stable oral care formulations comprising stannous salts, comprising higher levels of water while still preserving and even enhancing the antibacterial efficacy of the stannous ions.

### BRIEF SUMMARY

This disclosure provides oral care formulations with stable and effective concentrations of stannous ions, despite having relatively high levels of water. We have found that providing a reservoir of orthophosphate ion is effective to inhibit oxidation of stannous ion to stannic ion. This is surprising, because orthophosphate is generally a weak oxidizing agent. As the phosphorus is already in its highest oxidation state in orthophosphate ion, orthophosphate ion ordinarily would not act as a reducing agent or be expected to inhibit oxidation of stannous ion to stannic ion.

Without being bound by theory, the reservoir of orthophosphate ion may, for example, be provided by a phosphoric acid in free or orally acceptable salt form and/or by a polyphosphate under conditions (e.g., a high-water environment with an acid buffer) which permit partial hydrolysis of the polyphosphate to release orthophosphate. The stannous may be further stabilized by the presence of an organic acid buffer, e.g. citrate buffer comprising citric acid and trisodium citrate, and optionally sorbitol. Furthermore, in order for stannous to complex with phosphate, the zinc phosphate must first dissociate; a seemingly unlikely scenario considering the reasons outlined in this application. However, without being bound by theory, the data sets provided herein are significant in that, by calculation, they demonstrate the mechanism of zinc phosphate dissolving to provide stabilizing phosphate anions, and that the liberated phosphate anions are able to complex with stannous.

Without being bound by theory, polyphosphates do not inherently provide stabilization, but their hydrolytic or decomposition products may. Polyphosphate hydrolysis has been known and well-studied, and as polyphosphates undergo hydrolysis, free orthophosphate ions are generated. Again, without being bound by theory, if stannous oxidation is maintained through the presence and coordination of orthophosphate, and orthophosphate is a naturally occurring degradation process, then the toothpaste formulation itself can be considered a reservoir of phosphate ions. Over the lifetime of the product, it is possible that additional phosphate ions are released to counteract the natural tendency of stannous to oxidize to stannic ions.

In one embodiment, therefore, the disclosure provides a high water oral care composition, e.g., a toothpaste, comprising an orally acceptable carrier, a stannous ion source selected from stannous fluoride, stannous chloride, and combinations thereof, an organic acid buffer, and at least 25% of water by weight of the formulation, wherein the composition comprises a reservoir of orthophosphate products sufficient to stabilize stannous ions released from the stannous ion source, wherein less than 20%, preferably less than 10% of the stannous ions are converted to stannic ions over a period of 35 days, e.g. at 60° C.

In a particular embodiment, zinc phosphate (Zn₃(PO₄)₂) is used to inhibit the oxidation of stannous ion to stannic ion. This is counterintuitive give the very low solubility of zinc phosphate in water (Kₛₚ˜10⁻³⁵) and the fact that zinc phosphate is not known as an antioxidant. The stability of the formulation is further enhanced by sorbitol, citrate and pyrophosphate. As depicted on FIGS. 1 and 2, aqueous formulations comprising stannous fluoride, zinc phosphate, citric acid, tetrasodium pyrophosphate and sorbitol are more stable and provide higher levels of soluble stannous ion than formulations comprising other combinations, e.g., (i) stannous fluoride and zinc phosphate; (ii) stannous fluoride, zinc phosphate, and sorbitol; (iii) stannous fluoride, zinc phosphate, citric acid, and tetrasodium pyrophosphate; or (iv) stannous fluoride, zinc phosphate, citric acid, and sorbitol.

Thus, in a particular embodiment, the disclosure thus provides an oral care product, for example a toothpaste, comprising a stannous ion source selected from stannous fluoride, stannous chloride, and combinations thereof; zinc phosphate; a citrate ion source, e.g. selected from citric acid, trisodium citrate, potassium citrate, zinc citrate, and combinations thereof; a polyphosphate, e.g. selected from pyrophosphate, tripolyphosphate, and combinations thereof; sorbitol; and at least 25% water, by weight of the formulation.

The present disclosure further provides methods of using the compositions disclosed herein to clean the teeth, reduce bacterially-generated biofilm and plaque, reduce gingivitis, inhibit tooth decay and formation of cavities, and reduce dentinal hypersensitivity, comprising applying a composition of the invention to the teeth at least once daily.

The present disclosure further provides methods of making the compositions disclosed herein.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 shows the stability of the Sn(II) ion over the course of 35 days in the presence of zinc phosphate (Zn₃(PO₄)₂), TSPP, and trisodium citrate separately.

FIG. 2 shows a summary of the effects of various ingredient combination on a slurry of SnF₂, Zn₃(PO₄)₂, and water.

FIG. 3 shows the stability of stannous ions over a period of 35 days at 60° C., showing that the presence of orthophosphate ion inhibits the oxidation of stannous ion.

### DETAILED DESCRIPTION

The following description of the preferred embodiment(s) is merely exemplary in nature and is in no way intended to limit the invention, its application, or uses.

The present disclosure provides, in a first embodiment, a high water oral care composition (Composition 1), e.g., a toothpaste, comprising an orally acceptable carrier, a stannous ion source selected from stannous fluoride, stannous chloride, and combinations thereof, an organic acid buffer, and at least 25% of water by weight of the formulation, wherein the composition comprises a reservoir of orthophosphate sufficient to stabilize stannous ions released from the stannous ion source, wherein less than 20% of the stannous ions are converted to stannic ions over a period of 35 days at 60° C. For example, the disclosure provides
1.1. Composition 1 wherein less than 10% of the stannous ions are converted to stannic ions over a period of 35 days.
1.2. Any foregoing composition wherein the stannous ion source is stannous fluoride.
1.3. The foregoing composition wherein the stannous fluoride is present in an amount of ca. 0.454% by weight of the composition.
1.4. Any foregoing composition wherein the organic acid buffer is a citrate buffer, e.g., comprising citric acid and trisodium citrate.
1.5. Any foregoing composition wherein the reservoir of orthophosphate comprises orthophosphate provided by hydrolysis of a polyphosphate in situ.
1.6. Any foregoing composition wherein the reservoir of orthophosphate comprises orthophosphate provided by dissolution of an orally acceptable salt of phosphoric acid in situ.
1.7. The foregoing composition wherein the orally acceptable salt of phosphoric acid is a poorly soluble salt.
1.8. The foregoing composition wherein the salt of phosphoric acid is zinc phosphate.
1.9. Any forgoing composition comprising a zinc phosphate, wherein greater than 50% of total zinc in the composition is present as soluble zinc after storage at 13 weeks.
1.10. Any foregoing composition further comprising sorbitol.
1.11. Any foregoing composition formed by combining the following ingredients; a stannous ion source selected from stannous fluoride, stannous chloride, and combinations thereof; zinc phosphate; a citrate ion source; a polyphosphate; sorbitol; and at least 25% water, by weight of the formulation.
1.12. Any foregoing composition which is a toothpaste.
1.13. Any foregoing composition which is a composition according to Composition 1A, et seq.
1.14. Any foregoing composition, wherein the reservoir of orthophosphate provides one or more formal phosphate ions (i.e., by releasing the phosphate ion through dissociation of a parent complex salt) wherein the phosphate ion is provided by zinc phosphate, sodium phosphate, and/or phosphoric acid, either alone or in combination.

In a particular embodiment, the present disclosure provides an oral care product (Composition 1A), formed by combining the following ingredients;
a. a stannous ion source selected from stannous fluoride, stannous chloride, and combinations thereof;
b. zinc phosphate;
c. a citrate ion source;
d. a polyphosphate;
e. sorbitol; and
f. at least 25% water, by weight of the formulation.
   In further embodiments, the disclosure provides:
1A.1. Composition 1 wherein the stannous ion source comprises stannous fluoride.
1A.2. Any of the foregoing compositions, wherein the stannous ion source comprises 0.4 to 0.5%, e.g., about 0.454% stannous fluoride, by weight of the composition.
1A.3. Any of the foregoing compositions, wherein the stannous ion source comprises 0.4 to 0.5%, e.g., about 0.454% stannous fluoride, by weight of the composition, and additionally comprises stannous chloride.
1A.4. Any of the foregoing compositions wherein the citrate ion source is selected from citric acid, trisodium citrate, potassium citrate, zinc citrate, and combinations thereof.
1A.5. Any of the foregoing compositions wherein the citrate ion source is a combination of citric acid and trisodium citrate.
1A.6. Any of the foregoing compositions wherein the citrate ion source comprises a mixture of citric acid and trisodium citrate to provide a citrate ion content of ca. 0.8-0.9% by weight of the composition
1A.7. Any of the foregoing compositions wherein the polyphosphate is selected from alkali salts of pyrophosphate, tripolyphosphate, and combinations thereof.
1A.8. Any of the foregoing compositions, wherein the polyphosphate comprises tetrasodium pyrophosphate (TSPP).
1A.9. Any of the foregoing compositions, wherein the polyphosphate comprises ca. 2% tetrasodium pyrophosphate (TSPP) by weight of the composition.
1A.10. Any of the foregoing compositions wherein the molar ratio of the zinc phosphate to the stannous ion source is between 0.5:1 and 2:1, e.g., between 0.5:1 and 1:1, e.g., about 0.8:1.
1A.11. Any of the foregoing compositions wherein the zinc phosphate is present as zinc phosphate hydrate in an amount of ca. 1% by weight of the composition.
1A.12. Any foregoing composition wherein the sorbitol is present in an amount of ca. 22-30% by weight of the composition.
1A.13. Any foregoing composition wherein the water is present in an amount of water in an amount of ca. 28-35% by weight of the composition.
1A.14. Any of the foregoing compositions, wherein the composition comprises an effective amount of a silica abrasive, e.g., 20-30%, by weight of the composition.
1A.15. Any of the foregoing compositions
   (i) wherein the composition comprises an effective amount of a silica abrasive, e.g., 20-30%, by weight of the composition, wherein the silica abrasive is a mixture of a high cleaning silica having a PCR of ca. 95-105 using a 20% formulation, an RDA of ca. 170-190 using a 20% formulation, and a linseed oil absorption of ca. 50-70 ml/100 g and a second abrasive silica, having a PCR of ca. 75-85 using a 20% formulation, an RDA of ca. 80-100 using a 20% formulation, and a linseed oil absorption of ca. 80-100 ml/100 g; or
   (ii) wherein the composition comprises an effective amount of a silica abrasive, e.g., 20-30%, by weight of the composition, wherein the silica abrasive is a high cleaning silica having a PCR of ca. 95-105 using a 20% formulation, an RDA of ca. 170-190 using a 20% formulation, and a linseed oil absorption of ca. 50-70 ml/100 g.
1A.16. Any of the foregoing compositions comprising one or more surfactants, e.g., selected from anionic, cationic, zwitterionic, and nonionic surfactants, and mixtures thereof, e.g., comprising an anionic surfactant, e.g., a surfactant selected from sodium lauryl sulfate, sodium ether lauryl sulfate, and mixtures thereof, e.g. in an amount of from 0.3% to 4.5% by weight, e.g., 0.5 to 1, or 2 or 3 or 4%, e.g. 1-2% sodium lauryl sulfate (SLS), e.g., 1.5% SLS; and/or a zwitterionic surfactant, for example a betaine surfactant, for example cocamidopropylbetaine, e.g. in an amount of from about 0.1% to about 4.5% by weight, e.g. 0.5-2% cocamidopropylbetaine, e.g., about 1.25%.
1A.17. Any of the foregoing compositions comprising SLS.
1A.18. Any of the foregoing compositions comprising SLS in an amount of 1.5% by weight of the composition.
1A.19. Any of the foregoing compositions comprising cocamidopropylbetaine.
1A.20. Any of the foregoing compositions comprising cocamidopropylbetaine in an amount of 1.25% by weight of the composition
1A.21. Any of the foregoing compositions further comprising a viscosity modifying amount of one or more of polysaccharide gums, for example xanthan gum or carrageenan, carboxymethyl cellulose, silica thickener, and combinations thereof, e.g., xanthan gum.
1A.22. Any of the foregoing compositions further comprising xanthan gum in an amount of about 0.1% by weight of the composition.
1A.23. Any of the foregoing compositions comprising gum strips or fragments.
1A.24. Any of the foregoing compositions comprising polyethylene glycol, e.g., PEG 600.
1A.25. Any of the foregoing compositions further comprising flavoring, fragrance and/or coloring.
1A.26. Any of the foregoing compositions further comprising a whitening agent, e.g., a selected from the group consisting of peroxides, metal chlorites, perborates, percarbonates, peroxyacids, hypochlorites, and combinations thereof.
1A.27. Any of the foregoing compositions further comprising hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate);
1A.28. Any of the foregoing compositions further comprising a physiologically or orally acceptable potassium salt, e.g., potassium nitrate or potassium chloride, in an amount effective to reduce dentinal hypersensitivity.
1A.29. Any of the foregoing compositions further comprising a breath freshener, fragrance or flavoring.
1A.30. Any of the foregoing compositions, wherein the pH of the composition is from pH 5 to pH 8.5, for example, from pH 5 to 7.5, or 5 to 7, or 5.5 to 7.5, or 5.5 to 7, or 6 to 8, or 6 to 7.5, or 6.5 to 8, or 6.5 to 7.5, or 6 to 7, or 6.5 to 7, or 6 to 6.5, or about 6, or about 6.5 or about 7 or about 7.6 or about 8.2.
1A.31. Any foregoing composition which is a toothpaste.
1A.32. Any of the foregoing composition wherein the combination of ingredients in the composition results in formation of the following ions:
   Zn(C₆H₅O₇)⁻
   Zn(C₆H₅O₇)₂⁴⁻
   Zn(P₂O₇)₂⁶⁻
   Zn(P₂O₇)²⁻
   Zn(HPO₄)₂²⁻
   Sn(OH)(PO₄)²⁻
   SnPO₄⁻
   Sn(P₂O₇)₂⁻
1A.33. Any foregoing composition comprising a combination of zinc phosphate and zinc citrate.
1A.34. Any foregoing composition wherein, after at least one week of storage, substantially all the stannous ions are in the form of the Sn(OH)(PO₄)²⁻, SnPO₄⁻, Sn(P₂O₇)₂⁻, and over half of the zinc phosphate has been solubilized.
1A.35. Any foregoing composition wherein the composition comprises a reservoir of hydrolysis/dissociation products of zinc phosphate in sorbitol sufficient to stabilize stannous ions released from a mixture of stannous fluoride, citric acid and trisodium citrate in form of stannous phosphate compounds.
1A.36. Any foregoing composition wherein greater than 50% of total zinc in the composition is present as soluble zinc after storage at 13 weeks.
1A.37. Any foregoing composition wherein the conversion of stannous species to stannic species is less than 20%, e.g. less than 10% over a period of 35 days storage, e.g. at elevated temperature, e.g., at 60° C.
1A.38. Any foregoing composition having a formulation substantially in accordance with any of Formulations A, B, C, D, E, or F of Example 4 hereof, wherein by “substantially in accordance” is meant that the relative amounts of the ingredients are +/−10% of the ingredient amounts set forth (for clarity, “+/−10% of the ingredient amounts set forth” means that if an ingredient is listed as present in the amount of 20% by weight of the composition, it may be present in an amount of 18-22% by weight of the composition.)
1A.39. Any foregoing composition which comprises a combination of stannous fluoride, citric acid, zinc phosphate, sorbitol, tetrasodium pyrophosphate (TSPP), glycerin, polyethylene glycol (PEG 600) and gum(s).
1A.40. Any foregoing composition comprising
   a stannous ion source which is 0.454% stannous fluoride
   zinc phosphate in an amount of 1%;
   a citrate ion source comprising citric acid and trisodium citrate, to provide citrate ion content of 0.8-0.9%;
   a polyphosphate which is 2% TSPP;
   sorbitol, in an amount of ca. 22-30%; and
   water in an amount of ca. 28-35%;
   and optionally further comprising
   20-25% silica abrasive,
   thickeners (e.g., selected from xanthan gum, NaCMC, MCC,
   Gantrez, thickening silica, and combinations thereof),
   humectants in addition to sorbitol (e.g., selected from glycerin, PEG 600, propylene glycol, and combinations thereof),
   surfactants (e.g., comprising a combination of SLS and cocamidopropyl betaine),
   flavoring (including flavorants, cooling agents, and sweeteners), and/or
   pigment (e.g., titanium dioxide or a mixture of titanium dioxide and mica).
1A.41. Any foregoing composition comprising a reaction product of sequentially combining: an aqueous mixture comprising sorbitol and zinc phosphate; with an aqueous mixture comprising TSPP, glycerin, PEG 600 and gums; and then combining the product thus obtained with an aqueous mixture comprising stannous fluoride, citric acid and trisodium citrate.
1A.42. Any foregoing composition which is also a composition of any of Composition 1, et seq.
1A.43. Any of the foregoing toothpaste compositions for use to clean the teeth, reduce bacterially-generated biofilm and plaque, reduce gingivitis, inhibit tooth decay and formation of cavities, and/or reduce dentinal hypersensitivity.
1A.44. Any foregoing composition, further comprising sodium phosphate, and/or phosphoric acid.

In one embodiment, any of the compositions of Composition 1-A, et seq, comprise:
a stannous ion source which is 0.454% stannous fluoride
1-2.35% zinc phosphate hydrate;
a citrate ion source comprising citric acid and trisodium citrate, with total citrate ion content of ca. 0.8-0.9%;
a polyphosphate which is 2-4% TSPP;
sorbitol, in an amount of ca. 22-30%; and
water in an amount of ca. 28-35%, including the added water, and the water from the 70% sorbitol solution, and the Gantrez solution where present;
   together with 20-25% silica abrasive, thickeners (e.g., xanthan gum, NaCMC, MCC, Gantrez, and/or thickening silica), humectants in addition to sorbitol (e.g., glycerin, PEG 600, propylene glycol), surfactants (e.g., SLS and cocamidopropyl betaine), flavoring and pigment.

The present disclosure further provides methods to clean the teeth, reduce bacterially-generated biofilm and plaque, reduce gingivitis, inhibit tooth decay and formation of cavities, and reduce dentinal hypersensitivity, comprising applying an effective amount of a composition of the invention, e.g., any of Composition 1, et seq. or Composition 1A, et seq. to the teeth, and optionally then rinsing with water or aqueous solution.

For example, in various embodiments, the present disclosure provides methods to (i) reduce hypersensitivity of the teeth, (ii) to reduce plaque accumulation, (iii) reduce or inhibit demineralization and promote remineralization of the teeth, (iv) inhibit microbial biofilm formation in the oral cavity, (v) reduce or inhibit gingivitis, (vi) promote healing of sores or cuts in the mouth, (vii) reduce levels of acid producing bacteria, (viii) to increase relative levels of non-cariogenic and/or non-plaque forming bacteria, (ix) reduce or inhibit formation of dental caries, (x), reduce, repair or inhibit pre-carious lesions of the enamel, e.g., as detected by quantitative light-induced fluorescence (QLF) or electrical caries measurement (ECM), (xi) treat, relieve or reduce dry mouth, (xii) clean the teeth and oral cavity, (xiii) whiten teeth; (xiv) reduce tartar build-up, and/or (xv) promote systemic health, including cardiovascular health, e.g., by reducing potential for systemic infection via the oral tissues, comprising applying any of Compositions 1, et seq., as described above to the oral cavity of a person in need thereof, e.g., one or more times per day. The disclosure further provides e.g., any of Composition 1, et seq. or Composition 1A, et seq. for use in any of these methods.

The present disclosure further provides the use of an orthophosphate ion to inhibit oxidation of a stannous ion, e.g., in a composition according to any of Composition 1, et seq. or Composition 1A, et seq.

The present disclosure further provides methods of making the compositions disclosed herein, e.g., Composition 1, et seq., comprising
preparing
   a first aqueous premix comprising sorbitol and zinc phosphate
   a second aqueous premix comprising a polyphosphate; and one or more rheology-modifying agents (e.g., selected from polyalkylene glycols (e.g., PEG600), gums (e.g. xanthan gum), and combinations thereof); and
   a third aqueous premix comprising stannous fluoride and a citrate ion source;
adding the second aqueous premix to the first aqueous premix,
adding the third aqueous premix to the mixture of the first and second aqueous premixes, and
adding surfactant (e.g., sodium lauryl sulfate), abrasive agents (e.g. silica abrasive), sweetener and cooling agents.

The present disclosure further provides the product of the foregoing process.

Active Agents:

The compositions disclosed herein may comprise various agents which are active to protect and enhance the strength and integrity of the enamel and tooth structure and/or to reduce bacteria and associated tooth decay and/or gum disease, including or in addition to zinc and stannous materials. Effective concentration of the active ingredients used herein will depend on the particular agent and the delivery system used. It is understood that a toothpaste for example will typically be diluted with water upon use, while a mouth rinse typically will not be. The concentration will also depend on the exact salt or polymer selected. For example, where the active agent is provided in salt form, the counterion will affect the weight of the salt, so that if the counterion is heavier, more salt by weight will be required to provide the same concentration of active ion in the final product. Arginine, where present, may be present at levels from, e.g., about 0.1 to about 20 wt % (expressed as weight of free base), e.g., about 1 to about 10 wt % for a consumer toothpaste or about 7 to about 20 wt % for a professional or prescription treatment product. Fluoride where present may be present at levels of, e.g., about 25 to about 25,000 ppm, for example about 750 to about 2,000 ppm for a consumer toothpaste, or about 2,000 to about 25,000 ppm for a professional or prescription treatment product. Levels of antibacterial agents will vary similarly, with levels used in toothpaste being e.g., about 5 to about 15 times greater than used in mouthrinse. For example, a triclosan toothpaste may contain about 0.3 wt % triclosan.

In particular embodiments, the compositions comprise 1% zinc phosphate hydrate.

Fluoride Ion Source:

The oral care compositions may further include one or more fluoride ion sources, e.g., soluble fluoride salts. In some embodiments, fluoride is provided by stannous fluoride, but a wide variety of fluoride ion-yielding materials can be employed as alternative or additional sources of soluble fluoride in the present compositions. Representative alternative or additional fluoride ion sources include, but are not limited to, sodium fluoride, potassium fluoride, sodium monofluorophosphate, sodium fluorosilicate, ammonium fluorosilicate, amine fluoride, ammonium fluoride, and combinations thereof. In certain embodiments, the fluoride ion source includes stannous fluoride, sodium fluoride, sodium monofluorophosphate as well as mixtures thereof. In certain embodiments, the oral care composition of the invention may also contain a source of fluoride ions or fluorine-providing ingredient in amounts sufficient to supply about 25 ppm to about 25,000 ppm of fluoride ions, generally at least about 500 ppm, e.g., about 500 to about 2000 ppm, e.g., about 1000 to about 1600 ppm, e.g., about 1450 ppm. The appropriate level of fluoride will depend on the particular application. A toothpaste for general consumer use would typically have about 1000 to about 1500 ppm, with pediatric toothpaste having somewhat less. A dentifrice or coating for professional application could have as much as about 5,000 or even about 25,000 ppm fluoride. Fluoride ion sources may be added to the compositions of the invention at a level of about 0.01 wt. % to about 10 wt. % in one embodiment, or about 0.03 wt. % to about 5 wt. % in another embodiment, or about 0.1 wt. % to about 1 wt. % by weight of the composition in another embodiment. Weights of fluoride salts to provide the appropriate level of fluoride ion will obviously vary based on the weight of the counterion in the salt. In one embodiment, the composition contains sodium fluoride as a fluoride source in an amount of 0.03% to 5%, or 0.1% to 1% by weight of the composition, or about 0.32% by weight of the composition.

In particular embodiments herein, the fluoride ion source is provided by stannous fluoride, e.g. in an amount of about 0.454%.

Abrasives:

The compositions disclosed herein, may include silica abrasives, and may comprise additional abrasives, e.g., a calcium phosphate abrasive, e.g., tricalcium phosphate (Ca₃(PO₄)₂), hydroxyapatite (Ca₁₀(PO₄)₆(OH)₂), or dicalcium phosphate dihydrate (CaHPO₄·2H₂O, also sometimes referred to herein as DiCal) or calcium pyrophosphate; calcium carbonate abrasive; or abrasives such as sodium metaphosphate, potassium metaphosphate, aluminum silicate, calcined alumina, bentonite or other siliceous materials, or combinations thereof. These abrasives, generally have an average particle size ranging between about 1 and about 30 microns, about between 5 and about 15 microns. These particulate silica abrasives are distinct from colloidal silica thickeners.

Relative Dentin Abrasivity (RDA) is a measure of abrasivity, and the Pellicle Cleaning Ratio (PCR) is a measure of stain removal performance. Ordinary abrasive silica is a synthetic precipitated silica having a PCR in the range of 75-85 and RDA in the range of 80-100 when formulated at 20% of a toothpaste, and a linseed oil absorption of ca. 80-100 ml/100 g. Zeodent 113, available from Evonik, is a typical example. High cleaning silica is a harder, more abrasive silica, with a higher PCR (ca. 95-105) and RDA (ca. 170-190) and a lower linseed oil absorption (ca. 50-70 ml/100 g). Zeodent 103 (Evonik) is a typical example. In certain embodiments, the compositions comprise a mixture of ordinary abrasive silica and high cleaning silica, e.g., in a 1:1 mixture, e.g., 20-30% of a combination of ordinary abrasive silica and high cleaning silica. In other cases, substantially all of the abrasive silica is high cleaning silica, e.g., 20-30%, e.g., 25% high cleaning silica.

Foaming Agents:

The oral care compositions disclosed herein also may include an agent to increase the amount of foam that is produced when the oral cavity is brushed. Illustrative examples of agents that increase the amount of foam include, but are not limited to polyoxyethylene and certain polymers including, but not limited to, alginate polymers. The polyoxyethylene may increase the amount of foam and the thickness of the foam generated by the oral care carrier component of the present invention. Polyoxyethylene is also commonly known as polyethylene glycol (“PEG”) or polyethylene oxide. The polyoxyethylenes suitable for this invention will have a molecular weight of about 200,000 to about 7,000,000. In one embodiment the molecular weight will be about 600,000 to about 2,000,000 and in another embodiment about 800,000 to about 1,000,000. Polyox® is the trade name for the high molecular weight polyoxyethylene produced by Union Carbide. The polyoxyethylene may be present in an amount of about 1% to about 90%, in one embodiment about 5% to about 50% and in another embodiment about 10% to about 20% by weight of the oral care carrier component of the oral care compositions of the present invention. Where present, the amount of foaming agent in the oral care composition (i.e., a single dose) is about 0.01 to about 0.9% by weight, about 0.05 to about 0.5% by weight, and in another embodiment about 0.1 to about 0.2% by weight.

Surfactants:

The compositions disclosed herein may contain anionic surfactants, for example:
i. water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomonoglyceride sulfate,
ii. higher alkyl sulfates, such as sodium lauryl sulfate,
iii. higher alkyl-ether sulfates, e.g., of formula CH₃(CH₂)ₘCH₂(OCH₂CH₂)ₙOSO₃X, wherein m is 6-16, e.g., 10, n is 1-6, e.g., 2, 3 or 4, and X is Na or K, for example sodium laureth-2 sulfate (CH₃(CH₂)₁₀CH₂(OCH₂CH₂)₂OSO₃Na).
iv. higher alkyl aryl sulfonates such as sodium dodecyl benzene sulfonate (sodium lauryl benzene sulfonate)
v. higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate.

By “higher alkyl” is meant, e.g., C₆₋₃₀ alkyl. In particular embodiments, the anionic surfactant is selected from sodium lauryl sulfate and sodium ether lauryl sulfate. The anionic surfactant may be present in an amount which is effective, e.g., >0.01% by weight of the formulation, but not at a concentration which would be irritating to the oral tissue, e.g., <10%, and optimal concentrations depend on the particular formulation and the particular surfactant. For example, concentrations used or a mouthwash are typically on the order of one tenth that used for a toothpaste. In one embodiment, the anionic surfactant is present in a toothpaste at from about 0.3% to about 4.5% by weight, e.g., about 1.5%. The compositions of the invention may optionally contain mixtures of surfactants, e.g., comprising anionic surfactants and other surfactants that may be anionic, cationic, zwitterionic or nonionic. Generally, surfactants are those which are reasonably stable throughout a wide pH range. In certain embodiments, the anionic surfactants useful herein include the water-soluble salts of alkyl sulfates having about 10 to about 18 carbon atoms in the alkyl radical and the water-soluble salts of sulfonated monoglycerides of fatty acids having about 10 to about 18 carbon atoms. Sodium lauryl sulfate, sodium lauroyl sarcosinate and sodium coconut monoglyceride sulfonates are examples of anionic surfactants of this type. In a particular embodiment, the composition of the invention, e.g., Composition 1, et seq., comprises sodium lauryl sulfate.

The surfactant or mixtures of compatible surfactants can be present in the compositions of the present invention in about 0.1% to about 5.0%, in another embodiment about 0.3% to about 3.0% and in another embodiment about 0.5% to about 2.0% by weight of the total composition.

In a particular embodiment, the surfactants comprise an anionic surfactant, e.g., sodium lauryl sulfate, and a zwitterionic surfactant, e.g. cocamidopropyl betaine, e.g., ca. 1-2% sodium lauryl sulfate and 1-1.5% cocamidopropyl betaine.

Tartar Control Agents:

In various embodiments, the compositions disclosed herein may comprise an anticalculus (tartar control) agent. Suitable anticalculus agents include without limitation phosphates and polyphosphates (for example pyrophosphates), polyaminopropanesulfonic acid (AMPS), hexametaphosphate salts, zinc citrate trihydrate, polypeptides, polyolefin sulfonates, polyolefin phosphates, diphosphonates. The invention thus may comprise phosphate salts. In particular embodiments, these salts are alkali phosphate salts, i.e., salts of alkali metal hydroxides or alkaline earth hydroxides, for example, sodium, potassium or calcium salts. “Phosphate” as used herein encompasses orally acceptable mono- and polyphosphates, for example, P₁₋₆ phosphates, for example monomeric phosphates such as monobasic, dibasic or tribasic phosphate; dimeric phosphates such as pyrophosphates; and multimeric phosphates, e.g., sodium hexametaphosphate. In particular examples, the selected phosphate is selected from alkali dibasic phosphate and alkali pyrophosphate salts, e.g., selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these. In a particular embodiment, for example the compositions comprise a mixture of tetrasodium pyrophosphate (Na₄P₂O₇), calcium pyrophosphate (Ca₂P₂O₇), and sodium phosphate dibasic (Na₂HPO₄), e.g., in amounts of ca. 3-4% of the sodium phosphate dibasic and ca. 0.2-1% of each of the pyrophosphates. In another embodiment, the compositions comprise a mixture of tetrasodium pyrophosphate (TSPP) and sodium tripolyphosphate (STPP)(Na₅P₃O₁₀), e.g., in proportions of TSPP at about 1-2% and STPP at about 7% to about 10%. Such phosphates are provided in an amount effective to aid in cleaning the teeth, and/or to reduce tartar buildup on the teeth, for example in an amount of 2-20%, e.g., ca. 5-15%, by weight of the composition.

In a particular embodiment, the compositions comprise 2-4%, e.g., about 2%, of tetrasodium pyrophosphate.

Flavoring Agents:

The oral care compositions disclosed herein may also include one or more flavoring agents. Flavoring agents which are used in the practice of the present invention include, but are not limited to, essential oils as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint. The flavoring agent may be incorporated in the oral composition at a concentration of about 0.1 to about 5% by weight e.g. about 0.5 to about 1.5% by weight. Additionally, the formulations may comprise sweeteners, for example saccharin and/or sucralose.

Polymers:

The oral care compositions disclosed herein may also include additional polymers to adjust the viscosity of the formulation or enhance the solubility of other ingredients. Such additional polymers include polysaccharides (e.g., cellulose derivatives, for example carboxymethyl cellulose, or polysaccharide gums, for example xanthan gum or carrageenan gum), and polyvinyl pyrrolidone. Acidic polymers, for example polyacrylate gels, may be provided in the form of their free acids or partially or fully neutralized water soluble alkali metal (e.g., potassium and sodium) or ammonium salts.

Silica thickeners, which form polymeric structures or gels in aqueous media, may be present. Note that these silica thickeners are physically and functionally distinct from the particulate silica abrasives also present in the compositions, as the silica thickeners are very finely divided and provide little or no abrasive action. Other thickening agents are carboxyvinyl polymers, carrageenan, hydroxyethyl cellulose and water-soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate can also be used as component of the thickening composition to further improve the composition's texture. In certain embodiments, thickening agents in an amount of 0.5% to 5.0% by weight of the total composition are used.

The compositions disclosed herein may include an anionic polymer, for example in an amount of from about 0.05 to about 5%. Such agents are known generally for use in dentifrice, although not for this particular application, useful in the present invention are disclosed in U.S. Pat. Nos. 5,188,821 and 5,192,531; and include synthetic anionic polymeric polycarboxylates, such as 1:4 to 4:1 copolymers of maleic anhydride or acid with another polymerizable ethylenically unsaturated monomer, preferably methyl vinyl ether/maleic anhydride having a molecular weight (M.W.) of about 30,000 to about 1,000,000, most preferably about 300,000 to about 800,000. These copolymers are available for example as Gantrez. e.g., AN 139 (M.W. 500,000), AN 119 (M.W. 250,000) and preferably S-97 Pharmaceutical Grade (M.W. 700,000) available from ISP Technologies, Inc., Bound Brook, N.J. 08805. The enhancing agents when present are present in amounts ranging from about 0.05 to about 3% by weight. Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone. Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility. A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000. Another useful class of polymeric agents includes polyamino acids containing proportions of anionic surface-active amino acids such as aspartic acid, glutamic acid and phosphoserine.

The term average molecular weight of a polymer is the total weight of its sample divided by the number of molecules in the sample, i.e., ΣNᵢMᵢ/ΣNᵢ, and those skilled in the art can readily calculate the value using molecular weight values determined by size exclusion chromatograph or MALDI mass spectrometry.

In a particular embodiment, the thickening agents in the composition comprise xanthan gum, sodium carboxymethylcellulose (NaCMC), and microcrystalline cellulose (MCC).

Water:

The oral compositions may comprise significant levels of water. Water employed in the preparation of commercial oral compositions should be deionized and free of organic impurities. The amount of water in the compositions includes the free water which is added plus that amount which is introduced with other materials. In particular embodiments, the compositions contain 25-35% water.

Humectants:

Within certain embodiments of the oral compositions, it is also desirable to incorporate a humectant to prevent the composition from hardening upon exposure to air. Certain humectants can also impart desirable sweetness or flavor to the compositions. In addition to the sorbitol, suitable humectants may include other edible polyhydric alcohols such as polyethylene glycol, e.g., PEG 600, glycerin, xylitol, propylene glycol as well as other polyols and mixtures of these humectants. In one embodiment of the disclosure, the oral compositions contain one or more structuring humectants, wherein the structuring humectant comprise PEG, glycerin, and/or PG, which are present in an amount of 1-10% by weight of the composition. Preferably, PEG is present in an amount of 2-4% by weight of the composition and PG and/or glycerin in an amount of 3-5% by weight of the composition.

Other Optional Ingredients:

In addition to the above-described components, the embodiments of the compositions disclosed herein can contain a variety of optional dentifrice ingredients some of which are described below.

Unless stated otherwise, all percentages of composition components given in this specification are by weight based on a total composition or formulation weight of 100%.

It is understood that, in certain cases, an ingredient may perform multiple functions. For example, polyethylene glycol may affect the viscosity of the product, but may also act as a humectant; zinc salts may help stabilize the stannous, but may also provide antibacterial benefits; and stannous fluoride may serve as a source of both stannous ions and fluoride ions.

The compositions and formulations as provided herein are described and claimed with reference to their ingredients, as is usual in the art. As would be evident to one skilled in the art, the ingredients may in some instances react with one another, so that the true composition of the final formulation may not correspond exactly to the ingredients listed. Thus, it should be understood that the invention extends to the product of the combination of the listed ingredients.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

### EXAMPLES

### Example 1

### Formulation Design

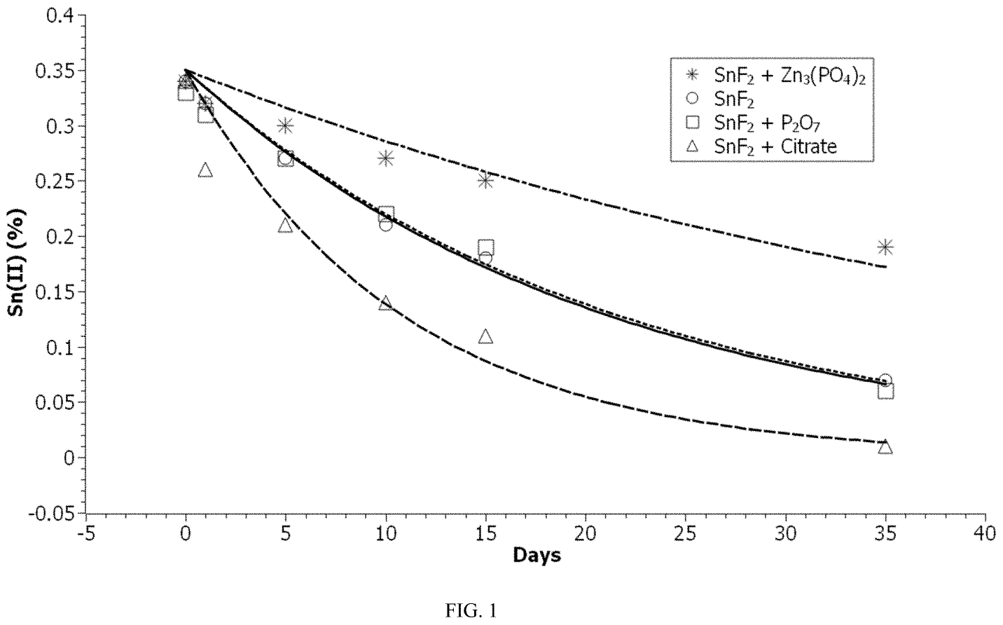

Stability of Sn(II) in aqueous solutions, and the ability of various additives to affect this stability, is assessed. FIG. 1 shows the stability of the Sn(II) ion over the course of 35 days in the presence of zinc phosphate (Zn₃(PO₄)₂), TSPP, and trisodium citrate separately. Over this time period, the addition of Zn₃(PO₄)₂ has a positive impact on the prevention of the stannous-to-stannic oxidation. Without being bound by theory, this is counterintuitive given the low solubility of this complex (Zn₃(PO₄)₂ Kₛₚ˜10⁻³⁵). These results are further unexpected given that Zn₃(PO₄)₂ is not generally considered an antioxidant.

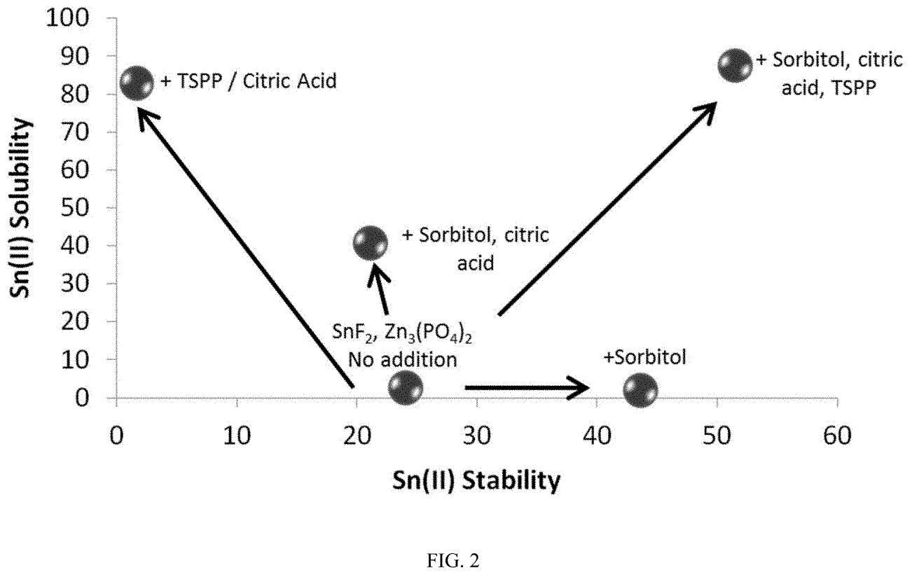

FIG. 2 shows a summary of the effects of various ingredient combination on a slurry of SnF₂, Zn₃(PO₄)₂, and water. Adding TSPP and citric acid to the Sn and Zn mixture significantly increases solubility but at a large compromise to Sn(II) stability. Adding sorbitol or sorbitol and citric acid produced mild improvements in stability and solubility, respectively. However, the addition of sorbitol, citric acid, and TSPP provided a large increase in solubility and stability. While sorbitol does provide a certain degree of stability, its effects alone are limited, and additional materials are necessary to increase this stability.

FIGS. 1 and 2 demonstrate that phosphate and sorbitol are needed to provide the greatest stability/solubility results, and while sorbitol can simply be added, the only phosphate source is zinc phosphate.

### Speciation Calculations

A series of speciation calculations is performed with particular consideration to other chelating agents in the formulation. The calculation evaluates a mixture of SnF₂, TSPP, Zn₃(PO₄)₂, trisodium citrate, and citric acid at their formulation concentrations and uses the formation constants (Table 1) of major Sn and Zn complexes to predict the major metal complexes resulting from this mixture. The results of this simulation are listed in Table 1. Comparison of starting materials of zinc phosphate and stannous fluoride, ˜56% of Zinc phosphate reacts to form other compounds while stannous fluoride is fully consumed by the reaction. The next five most abundant zinc complexes are comprised of ligands citrate, pyrophosphate, and a single ortho phosphate. In consideration of stannous fluoride, 95% of the most abundant stannous compounds are bound with a phosphate ligand, and the next most prevalent species is a stannous-pyrophosphate complex.

TABLE 1
Solid compounds based on 100 g of material
before and after reaching equilibrium

| Compound | Starting Moles | Ending Moles |
|---|---|---|
| Zinc phosphate | 2.31e−3 | 1.021e−3 |
| Zinc(citrate)⁻ | 0 | 1.29−3 |
| Zinc(citrate)₂⁴⁻ | 0 | 1.14e−3 |
| Zinc(pyro)₂⁶⁻ | 0 | 7.470e−4 |
| Zn(pyro)²⁻ | 0 | 5.029e−4 |
| Zn(HPO₄)₂²⁻ | 0 | 1.289e−4 |
| SnF₂ | 2.9e−3 | 0 |
| Sn(OH)(PO₄)²⁻ | 0 | 2.69e−4 |
| SnPO₄⁻ | 0 | 2.589e−4 |
| Sn(pyro)₂⁻ | 0 | 1.22e−5 |

In the formulation, the phosphate provides a stabilizing effect for stannous and zinc, and gives an added tartar control benefit. It is unexpected than an “insoluble” zinc salt can be dissociated over 50% in aqueous solution. Moreover, zinc phosphate is not considered an antioxidant and, therefore, has not been considered as a stannous stabilizing compound.

### Example 2

### Stabilizing Stannous Ions

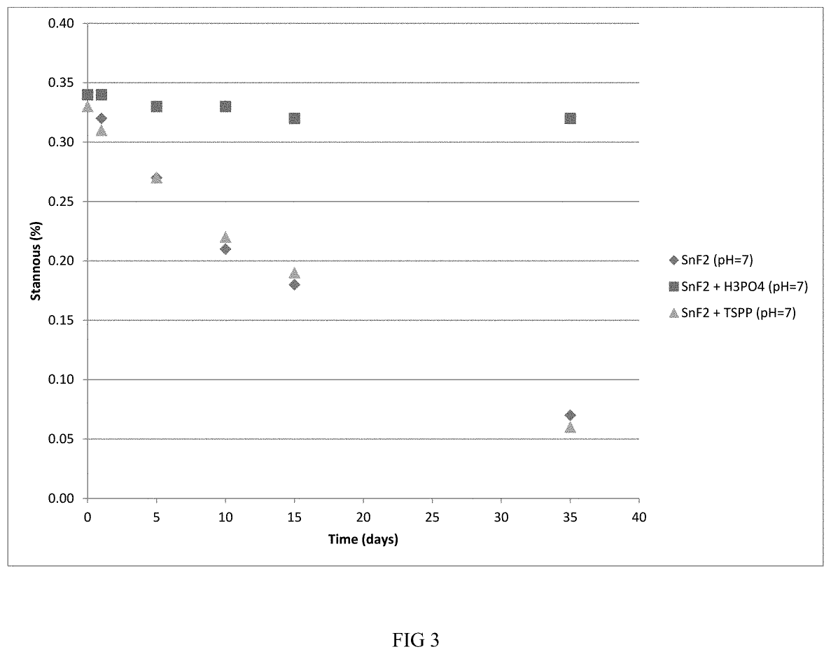

Stability of stannous ions is assessed over a period of 35 days. FIG. 3 shows the stability of stannous ions over a period of 35 days at 60° C. Over the course of this time, stannous fluoride oxidizes significantly by ˜80%. In the presence of phosphoric acid, only 8-9% oxidizes, indicating that orthophosphate ions are effective at stabilizing the Sn(II) oxidation state. Adding phosphoric acid, sodium phosphate or zinc phosphate into the formulation provides a formal phosphate ion that can be released through dissociation of the parent complex salt:

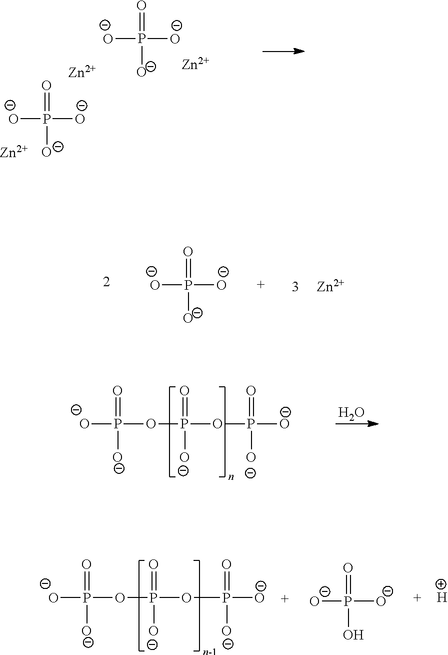

In contrast to orthophosphate, polyphosphates do not provide significant oxidative stability. FIG. 3 also shows SnF₂ in the presence of TSPP. While these two compounds coordinate, no oxidative stability is observed.

### Example 3

### Making the Formulation

The general process of making the formulation is carried by making the following aqueous mixtures:

Solution 1: Sorbitol, Zinc Phosphate

Solution 2: TSPP, glycerin, PEG600, gums

Solution 3: Stannous fluoride, citric acid, trisodium citrate

Solution 2 is added to Solution 1. Solution 3 is added to the mixture of 1 and 2. The resulting mixture is then combined with surfactants and remaining ingredients.

Combining a zinc phosphate+sorbitol mixture with TSPP, glycerin, PEG600 and the gums to form a combination of zinc phosphate and TSPP increases the solubility of Zn²⁺ from 0.0001% to 0.12% (by mass), indicating an initial reaction occurs, serving to dissociate the complex. In solution 3, stannous fluoride combines with citrate to likely form a variety of stannous-citrate complexes. When the combined Solutions 1 and 2 are mixed with Solution 3, the free phosphates are now able to complex with stannous, with the sorbitol providing an additional stabilizing feature. It is possible that zinc phosphate continues to dissociate overtime as it reacts with the excess citrate in the formulation. This may provide a continually increasing concentration of stabilizing phosphates that could maintain the stannous oxidation state over a longer period of time.

### Example 4: Exemplary Formulations

Test formulations are prepared with the following ingredients:

| INGREDIENTS (by wt %) | A | B | C | D | E | F |
|---|---|---|---|---|---|---|
| STANNOUS FLUORIDE | 0.454 | 0.454 | 0.454 | 0.454 | 0.454 | 0.454 |
| TETRASODIUM PYROPHOSPHATE | 2 | 2 | 2 | 2 | 2 | 4 |
| (TSPP) | | | | | | |
| NON-CRYSTAL SORBITOL | 42.45 | 39.1 | 36.346 | 36.876 | 32.5 | 38.45 |
| (70% aq. soln.) | | | | | | |
| DEMINERALIZED WATER | Q.S. (e.g., | Q.S. (e.g., | Q.S. (e.g., | Q.S. (e.g., | Q.S. (e.g., | Q.S. (e.g., |
| | 18-21%) | 18-21%) | 17-20%) | 17-20%) | 13-17%) | 13-17%) |
| ABRASIVES | 20 | 20 | 25 | 25 | 20 | 20 |
| THICKENERS | 3.6 | 3.6 | 2.6 | 2.6 | 14.6 | 9.6 |
| FLAVOR, SWEETENER, COLOR | 2.25 | 2.19 | 2.3 | 2.12 | 2.85 | 2.25 |
| ANIONIC SURFACTANT | 1.5 | 1.5 | 1.5 | 1.5 | 1.5 | 1.5 |
| ZWITTERIONIC SURFACTANT | 1.25 | 1.25 | 1.25 | 1.25 | 1.25 | 1.25 |
| ZINC PHOSPHATE HYDRATE | 1 | 1 | 1 | 1 | 2.35 | 1 |
| TRISODIUM CITRATE DIHYDRATE | 1 | 1 | 1 | 1 | 1 | 1 |
| CITRIC ACID - ANHYDROUS | 0.2 | 0.2 | 0.2 | 0.2 | 0.2 | 0.2 |
| HUMECTANTS IN ADDITION | 6 | 8.35 | 8.35 | 8 | 6 | 6 |
| TO SORBITOL | | | | | | |
| TOTAL | 100 | 100 | 100 | 100 | 100 | 100 |

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the scope of the invention should be construed broadly as set forth in the appended claims.

1. A high water oral care composition comprising
a. an orally acceptable carrier,
b. a stannous ion source selected from stannous fluoride, stannous chloride, and combinations thereof,
c. an organic acid buffer, and
d. at least 25% of water by weight of the formulation,
wherein the composition comprises a reservoir of orthophosphate sufficient to stabilize stannous ions released from the stannous ion source, wherein less than 20% of the stannous ions are converted to stannic ions over a period of 35 days at 60° C.

2. The composition of claim 1 wherein less than 10% of the stannous ions are converted to stannic ion over a period of 35 days at 60° C.

3. The composition of claim 1 wherein the stannous ion source is stannous fluoride.

4. The composition of claim 1 wherein the organic acid buffer is a citrate buffer comprising citric acid and trisodium citrate.

5. (canceled)

6. The composition of claim 1 wherein the reservoir of orthophosphate comprises orthophosphate provided by hydrolysis of a polyphosphate in situ.

7. The composition of claim 1 wherein the reservoir of orthophosphate comprises orthophosphate provided by dissolution of an orally acceptable salt of phosphoric acid in situ.

8. The composition of claim 7 wherein the orally acceptable salt of phosphoric acid is zinc phosphate.

9. The composition of claim 8 wherein greater than 50% of total zinc in the composition is present as soluble zinc after storage at 13 weeks.

10. The composition of claim 1 formed by combining the following ingredients; a stannous ion source selected from stannous fluoride, stannous chloride, and combinations thereof zinc phosphate; a citrate ion source; a polyphosphate; sorbitol; and at least 25% water, by weight of the formulation.

11. The composition of claim 1, wherein the reservoir of orthophosphate provides a formal phosphate ion, and wherein the phosphate ion is provided by zinc phosphate, sodium phosphate, and/or phosphoric acid, either alone or in combination.

12. An oral care composition formed by combining the following ingredients;
a. a stannous ion source selected from stannous fluoride, stannous chloride, and combinations thereof;
b. zinc phosphate;
c. a citrate ion source;
d. a polyphosphate;
e. sorbitol; and
f. at least 25% water, by weight of the composition.

13. A composition according to claim 12, wherein the stannous ion source comprises stannous fluoride.

14. A composition according to claim 12 wherein the citrate ion source comprises a mixture of citric acid and trisodium citrate.

15. A composition according to claim 12 wherein the polyphosphate is selected from alkali salts of pyrophosphate, tripolyphosphate, and combinations thereof.

16. A composition according to claim 12, wherein the polyphosphate comprises tetrasodium pyrophosphate (TSPP).

17. A composition according to claim 12 wherein the molar ratio of the zinc phosphate to the stannous ion source is between 0.5:1 and 2:1.

18. A composition according to claim 12 wherein the zinc phosphate is present as zinc phosphate hydrate in an amount of ca. 1% by weight of the composition.

19. A composition according to claim 12 wherein the sorbitol is present in an amount of ca. 22-30%, or ca. 28-35%, by weight of the composition.

20. (canceled)

21. (canceled)

22. (canceled)

23. A composition according to claim 1, wherein the combination of ingredients in the composition results in formation of the following ions:
Zn(C₆H₅O₇)⁻
Zn(C₆H₅O₇)₂⁴⁻
Zn(P₂O₇)₂⁶⁻
Zn(P₂O₇)²⁻
Zn(HPO₄)₂²⁻
Sn(OH)(PO₄)²⁻
SnPO₄⁻
Sn(P₂O₇)²⁻

24. A composition according to claim 1 wherein substantially all the stannous ions are in the form of the Sn(OH)(PO4)2-, SnPO4-, Sn(P2O7)2-, and over half of the zinc phosphate has been solubilized.

25. A composition according to claim 1, wherein the composition comprises a reservoir of hydrolysis/dissociation products of zinc phosphate in sorbitol sufficient to stabilize stannous ions released from a mixture of stannous fluoride, citric acid and trisodium citrate in form of stannous phosphate compounds.

26. A composition according to claim 1 wherein the composition comprises zinc phosphate and greater than 50% of total zinc in the composition is present as soluble zinc after storage at 13 weeks.

27. A composition according to claim 1 wherein the conversion of stannous species to stannic species is less than 10% over a period of 35 days storage.

28. A composition according to claim 1 which comprises
a. a stannous ion source which is 0.454% stannous fluoride
b. zinc phosphate hydrate in an amount of 1%;
c. a citrate ion source comprising citric acid and trisodium citrate, to provide citrate ion content of 0.8-0.9%;
d. a polyphosphate which is 2% TSPP;
e. sorbitol, in an amount of ca. 22-30%; and
f. water in an amount of ca. 28-35%;
and optionally further comprises
g. 20-25% silica abrasive,
h. thickeners,
i. humectants in addition to sorbitol,
j. surfactants,
k. flavoring and/or pigment,
wherein all percentage amounts are by weight of the composition.

29. A composition according to claim 1 comprising a reaction product of sequentially combining: an aqueous mixture of sorbitol and zinc phosphate; with an aqueous mixture of TSPP, glycerin, PEG 600 and gums; and then combining the product thus obtained with an aqueous mixture of stannous fluoride, citric acid and trisodium citrate.

30. (canceled)

31. A method to clean the teeth, reduce bacterially-generated biofilm and plaque, reduce gingivitis, inhibit tooth decay and formation of cavities, and reduce dentinal hypersensitivity, comprising applying an effective amount of a composition according to claim 1, and optionally then rinsing with water or aqueous solution.

32. (canceled)
